(19) United States
(12) Patent Application Publication
(10) Pub. No.: US 2019/0374009 A1
(43) Pub. Date: Dec. 12, 2019

(54) DIAPER PAD FANNY PACK

(71) Applicant: Kazumi Arikawa, La Crescenta, CA (US)

(72) Inventor: Kazumi Arikawa, La Crescenta, CA (US)

(21) Appl. No.: 16/433,949

(22) Filed: Jun. 6, 2019

Related U.S. Application Data
(60) Provisional application No. 62/682,285, filed on Jun. 8, 2018.

(51) Int. Cl. A45F 3/00; A45F 3/14

(52) U.S. Cl. CPC A45F 3/005; A61F 2013/15008; A45F 2003/144; A45F 3/14

(57) ABSTRACT

A diaper pad fanny pack has a diaper pad that is folded into a fanny pack and is positioned on a user to wear handsfree such as on a hip or over the shoulder. Fold lines positioned long the pad allow a user to fold the pad into a pack. When in an open position, the pad is unrolled and laid flat. There are compartments on the outer and inner surface of the diaper pad fanny pack to hold items such as diapers, wipes, change of clothes and sanitizers.

### CROSS-REFERENCE TO RELATED APPLICATIONS

The present application claims priority to U.S. Patent Application Ser. No. 62/682,285, filed Jun. 8, 2018, which is incorporated by reference herein in its entirety for all purposes.

### FIELD OF THE INVENTION

The present invention relates to child diaper changing pads. More particularly, the invention relates to a diaper changing pad that, when folded and in a closed position, may be worn on an adult's body hands-free.

### BACKGROUND OF THE INVENTION

The number of products geared to new parents or guardians of a baby is copious and constantly increasing. However, despite the number of products that exists in the world of baby and child products improvements are still needed. For instance, when the need to change a diaper arises, childcare givers normally have a diaper pad stuffed in a large baby bag that includes wipes and ointments but also a change of clothes, hand sanitizer, bottles, formula, etc. If a guardian needs to change a soiled diaper, the guardian must carry the child and everything that comes along in the fully-stocked diaper bag. Even if the guardian fills the diaper bag with the bare minimum the hag must still be toted along.

Diaper pads exist with compartments to hold diapers, wipes and ointments, however none exist that are portable by being toted hands-free. Currently, diaper pads exist that are part of a normally bulky diaper bag. Diaper pad also exist that have a loop to wear the diaper pad as a sort of “bracelet.” However, these “bracelet” diaper pads, would not allow a parent/guardian to attend to a child without a deadweight dangling from the user's wrist. The dangling diaper pad “bracelet” would get twisted around the user's wrist and obstruct a user from freely using their hands to care for a child. Bracelet-type diaper pads flail around posing a hitting risk to the child and the user or anyone near the unwieldy bracelet-type diaper pad.

Improvements need to products for babies, infants and toddlers so parents/guardians may attend to their child without obstructions to their hands. A need exists for a diaper pad that can be toted hands-free. A need also exists for a diaper pad that is not bulky. A need exists for a diaper pad that has compartments for holding items necessary in changing a soiled diaper and is also compact and can be worn on the user's body as a bag, a bandolero or a fanny pack.

### BRIEF SUMMARY

A diaper pad fanny pack is shown with a diaper pad having a length and fold lines disposed on the length of said pad so the pad may be folded on the fold lines and form a closed diaper pad position. There are wings disposed at sides of said length of said diaper pad and at least one strap having a first end and a second end. The first ends of each strap are affixed to one of said wings. In use, a user positions the folded diaper pad fanny pack around one's waist and fastens mating male and female fasteners on second ends of straps to place said pad, motionless, on the user.

When the diaper pad fanny pack is open, an inner surface is exposed which has a sanitary coat to be wipeable. There is at least one compartment on the inner surface. The compartment is a pocket and is dimensioned and configured to receive wipes, diapers or a change of clothes. Compartments may also be disposed on the outer surface of the diaper pad fanny pack.

### DESCRIPTION OF THE DRAWINGS

FIG. 1 shows a diaper pad fanny pack of the present invention in a closed position.

FIG. 2A shows the diaper pad fanny pack shown in FIG. 1 with straps open.

FIG. 2B shows the front and back views of the diaper pad fanny pack shown in FIG. 1 with straps open.

FIG. 3A shows the inside of the diaper pad fanny pack of the present invention in an open position with straps hidden.

FIG. 3B shows the outside and inside views of the diaper pad fanny pack of the present invention in the open position.

FIG. 4 shows another view similar to FIG. 1.

FIG. 5 shows another view similar to FIG. 3A with accoutrements placed in pockets.

FIG. 6 shows another view similar to FIG. 3A with accoutrements placed in pockets.

FIG. 7 shows another view similar to FIG. 3A.

### DESCRIPTION OF THE INVENTION

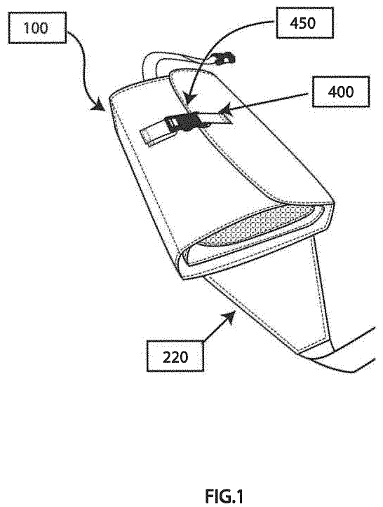

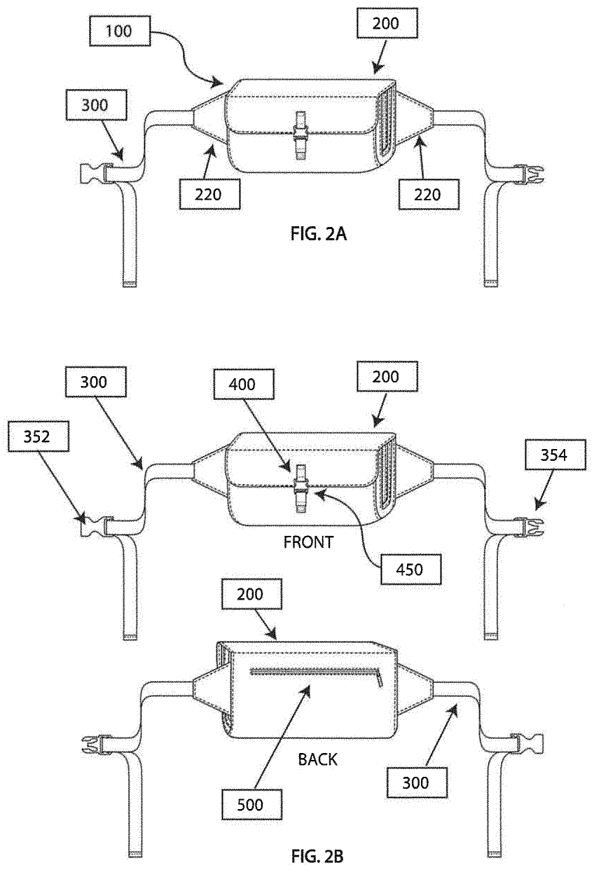

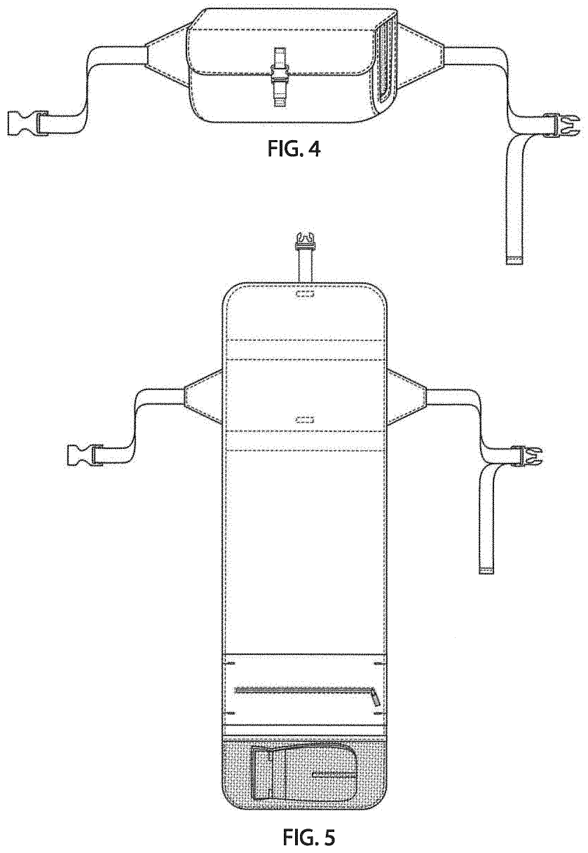

The diaper pad fanny pack 100 of the present invention is generally shown in FIG. 1. See also FIG. 4. The diaper pad fanny pack 100 has a main body 200 and straps 300 depending from wings 220. See FIGS. 2A and 2B. The wings 220 are positioned at each side of the pack 100, specifically at the horizontal sides of the pack 100. The wings 220 are placed so the straps 300 will secure the pack 100 on a user in a stationary manner. With the straps 300 securing the pack 100 in a horizontal position the pack 100 will not move or flail around. Alternative embodiments may exist in ways to secure more than one side of the pack 100 to a user.

The straps 300 are shown having two parts that employs fastener 350 comprising parts 352, 354. Female fastener portion 352 and male fastener portion 354 are mating. Fastener 450 is shown in a closed position on the face of the body 200. On the rear of the body 200 is a pocket 520. Pocket 520 is shown with a zipper fastener, but may be closed in any other manner or left open. In one embodiment, pocket 520 may be a small zippered compartment that is used to hold keys, money or a wallet.

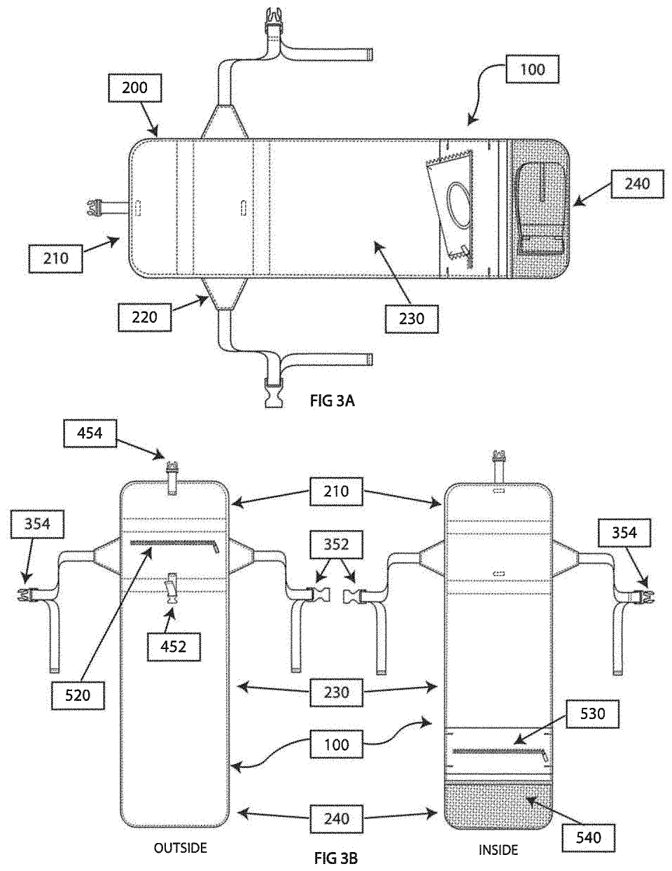

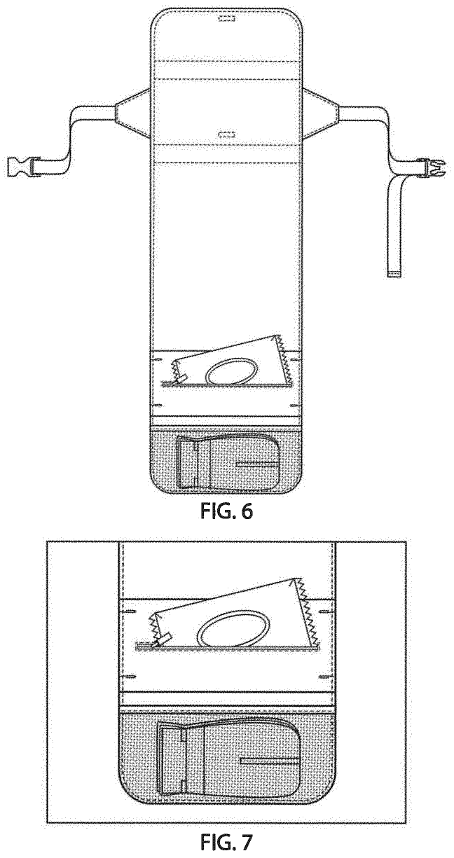

FIGS. 3A, 3B, and 5-7, show the pack 100 in an unfolded, open position. In this position, fastener 450 is shown detached exposing female fastener portion 452 and male fastener portion 454. Fasteners 452 and 454 are mating. Strap 400 is used with the fastener 450. In the open position an outer surface shows the fastener parts 452, 454 and pocket 520. An inner surface of the pack 100 shows first end 210, wings 220 which holds straps 300, pad body 230 and second end 240. Pockets 530 and 540 are shown at the second end 240 on the inner surface. Pocket 530 is a zippered pocket and pocket 540 is a mesh pocket. Collectively, pockets 520, 530, 540 are storage units or compartments 500 for retaining diapers, changing needs such as wipes, ointment, hand sanitizer as well as other accessories such as keys.

Pack 100 is a portable childcare accessory in that the straps 300 allows a user to hold the pack hands-free. See FIG. 4. The pack 100 provides a comfortable, minimal design with padding for a baby, but without the bulk of a traditional diaper bag. The pack 100 provides a minimal way to carry just the essentials for an active day with a child.

The pack 100 can be made of a coated, sanitary, wipeable diaper changing pad integrated with compartments 500 for wipes, diapers and a change of clothes. The pack 100 can be folded and comfortably worn on the waist, over a single shoulder or worn as a bandolero, across the chest.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A diaper pad fanny pack comprising:
a diaper pad having a length,
fold lines disposed on the length of said pad, wherein said pad may be folded on said fold lines forming a closed diaper pad position;
wings, wherein said wings are disposed at sides of said length of said diaper pad; and
at least one strap having a first end and a second end, each said first ends of said strap being affixed to one of said wings, wherein said strap may be dimensioned and configured to place said pad, motionless, on a user.

2. The diaper pad fanny pack of claim 1, wherein the pack exposes an inner surface when in an open position.

3. The diaper pad fanny pack of claim 2, wherein said inner surface has a sanitary coat and is wipeable.

4. The diaper pad fanny pack of claim 2, further comprising at least one compartment on said inner surface.

5. The diaper pad fanny pack of claim 4, wherein the pocket is dimensioned and configured to receive wipes.

6. The diaper pad fanny pack of claim 4, wherein the pocket is dimensioned and configured to receive diapers.

7. The diaper pad fanny pack of claim 4, wherein the pocket is dimensioned and configured to receive a change of clothes.

8. The diaper pad fanny pack of claim 1, further comprising at least one compartment on an outer surface of the pack.

9. The diaper pad fanny pack of claim 1, wherein said strap includes mating fasteners disposed at each said second ends of the strap.
